(19) United States
(12) Patent Application Publication
(10) Pub. No.: US 2019/0133774 A1
(43) Pub. Date: May 9, 2019

(54) Tibial Component for a Knee Endoprosthesis with Two PE Liners

(71) Applicant: CeramTec GmbH, Plochingen (DE)

(72) Inventors: Philipp DOLD, Esslingen (DE); Lotte FERNENGEL, Nürtingen (DE); Matthias LINDE, Esslingen (DE); Jens SIEGFRIED, Lübeck (DE); Guillaume BUGNARD, Solothurn (CH)

(21) Appl. No.: 16/096,797

(22) PCT Filed: Apr. 25, 2017
(86) PCT No.: PCT/EP2017/059686

§ 371 Date: Oct. 26, 2018

(30) Foreign Application Priority Data
Apr. 27, 2016 (DE) 10 2016 207 206.4

(51) Int. Cl. A61F 2/38

(52) U.S. Cl. CPC A61F 2/3868; A61F 2002/30377

(57) ABSTRACT

The invention relates to a tibial component (1) of a knee endoprosthesis for anchoring on the proximal tibia, comprising a tibial plateau (2), a tibial shaft (3) on the underside of the tibial plateau (2) facing the tibia, and having a liner (4) for receiving and supporting the lateral condyle surface of a femoral part.

To allow different heights and shapes of the liner to be provided medially and laterally, and to ensure that just one individual liner can be exchanged in the event of revision and, for this purpose, a minimally invasive intervention (operation) is sufficient, it is suggested according to the invention that the tibial component (1) has one liner or preferably two liners (4a, 4b) embodied as individual parts, which liner or liners is/are exchangeably anchored on the tibial component (1).

The invention relates to a tibial component of a knee endoprosthesis for anchoring on the proximal tibia, comprising a tibial plateau, a tibial shaft on the underside of the tibial plateau facing the tibia, and having a liner for receiving and supporting the lateral condyle surface of a femoral part.

The underlying object of the invention is to refine a tibial component according to the preamble of claim 1 to allow different heights and shapes of the liner to be provided medially and laterally. With respect to the shapes of medially pivoting designs, this includes different liner congruences for corresponding indication positions (preserving or replacing posterior cruciate ligament), weight bearing positions (A-P position), and a patient-specific design. A-P position is an abbreviation for anterior-posterior positioning.

According to the invention, this object is attained using a tibial component according to the features of claim 1.

Since the tibial component has one or, preferably, two liners embodied as individual parts that is/are exchangeably anchored on the tibial component, it is possible to provide different heights and shapes of the liners medially and laterally. If there is a revision, just one individual liner can be exchanged, as well. Due to the size of the liner, a minimally invasive intervention (operation) is sufficient. If necessary, an axis correction may also be performed with different liner heights.

During the operation, the surgeon may decide which liner he will use. He may select the best-fitting liner for this patient from a set of different liners, or, using 3-D printers, he may produce the best-fitting liner prior to or even during the operation.

The liners preferably comprise UHMWPE, crosslinked PE, vitamin E PE, PEEK, or PAEK. Vitamin E PE is a stabilized polyethylene liner that stabilizes free radicals in highly crosslinked polyethylene.

The tibial plateau and the tibial shaft preferably comprise a metal or a sintered ceramic or even a polymer.

Two preferred embodiments of the inventive tibial components are described in the following.

In a first embodiment, for each liner a recess is arranged on the upper side of the tibial plateau facing the femur part, in which recess the liner is exchangeably anchored. The advantage is found in the few components and thus fewer work steps for production. In addition, fixation is improved. No additional interface is required and thus there is no potential additional wear.

In a second embodiment, a back-molded polymer is arranged on the upper side of the tibial plateau facing the femur part, on which back-molded polymer the liners are each exchangeably anchored in a recess. Implantation (handling) is easier in contrast to the embodiment without polymer. No complex geometries (anchoring mechanism) are required in the ceramic. Because of this, lower costs are attained due to the ceramic components being less complex. In addition, greater strength is achieved due to the geometries being less complex.

In this second embodiment, the circumferential edge of the tibial plateau preferably has a notch in which a fitted catch of the back-molded polymer engages. This provides secure anchoring of the back-molded polymer on the tibial plateau. Back-molded polymer is also simply referred to as polymer in the following for reasons of simplicity.

In the two variants, the liners are preferably anchored in the recesses on the tibial plateau or on the polymer using a snap-fit or press-fit connection and also form-fit. Snap-fit and press-fit connections are sufficiently durable, even over a long period of time, and may also be detached again during a revision.

The snap-fit connection is preferably characterized in that the edge of each recess has a circumferential undercut and each liner has a snap-fit lip fitted to the undercut.

The arrangement where the snap-fit lip or undercut is arranged may also be reversed, of course. Both variants may be used both for a bicondylar and a unicondylar or revision tibial base plate. Only one liner, either medial or lateral, is used in the case of the unicondylar tibial base plate. Occasionally the liners for the bicondylar tibial base plate may also be used for the unicondylar tibial base plate, and thus an integrated modular knee system is used for the unicondylar and bicondylar intervention (depending on the revision).

For the revision, a ramp like a slit is preferably arranged on the tibial plateau or on the polymer for each recess, the ramp leading from the edge of the tibial plateau or of the polymer up to the floor of the recess, this making it possible to guide the tip of a lever tool, known per se, under the liner. A lever tool may be embodied like a golf club.

The invention relates to providing a three-part tibial component, also called “fixed bearing systems,” having two liners for a ceramic/metal tibial plateau. Two separate PE liners, UHMWPE, crosslinked PE, vitamin E PE, or other liner materials suitable for the knee endoprosthesis may be provided medially/laterally. The liners are preferably fixed in the tibial plateau using an undercut (snap-fit lip). The undercut may be provided both in the tibial plateau and in the additional polymer with which the tibial plateau was previously coated.

If a two-part liner is used, different PE height and PE shapes may be provided medially and laterally. The PE shapes include medially pivoting designs, different liner congruences for corresponding indications (preserving or replacing posterior cruciate ligament), weight-bearing position (A-P position), and patient specific designs. It is also possible to use only the medial or lateral liner for a corresponding unicondylar tibial base plate in the case of unicondylar care. In the case of a revision, the PE liners are lifted out of the tibial plateau or coated polymer using a ramp having an anterior/central orientation.

The invention shall be explained further using figures.

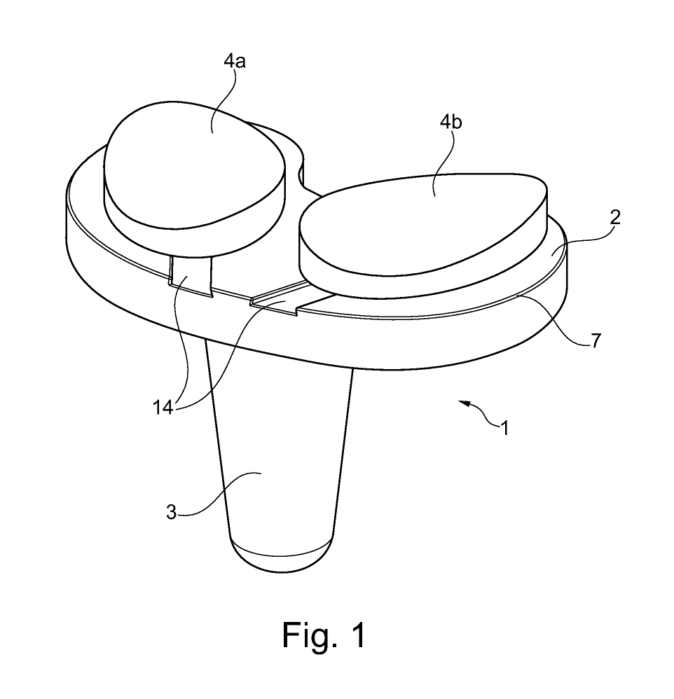

FIG. 1 depicts a first embodiment of an inventive tibial component 1 for anchoring the proximal tibia. This tibial component 1 comprises a tibial plateau 2, a tibial shaft 3, for anchoring in the tibia on the underside of the tibial plateau 2 facing the tibia, and two liners 4a, 4b for receiving and supporting the lateral condyle surfaces of a femoral part (not shown). The tibial plateau 2 and the tibial shaft 3 comprise a metal or a sintered ceramic or even polymer. The tibial component 1 has two liners 4a, 4b, embodied as individual parts, that are exchangeably anchored on the tibial component 1. For the revision, i.e., for exchanging the liners 4a, 4b, for each liner 4a, 4b a ramp 14, like a type of slit, is arranged on the tibial plateau 2, the ramps 14 leading from the edge 7 of the tibial plateau 2 to under the liner 4, 4b. This makes it possible to guide the tip of a lever tool, known per se, under the liner 4a, 4b.

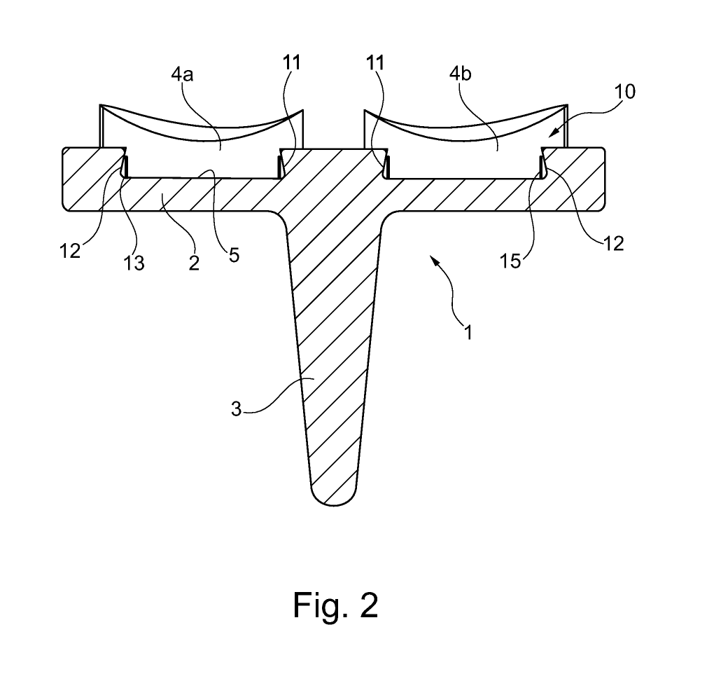

FIG. 2 depicts the tibial component 1 according to FIG. 1 in section. For anchoring the liners 4a, 4b, arranged on the upper side of the tibial plateau 2 facing the femur are two recesses 5 in which the liners 4a, 4b are exchangeably anchored. In the embodiment depicted, the liners 4a, 4b are anchored in the recesses 5 on the tibial plateau 2 using a snap-fit connection 10.

To this end, the edge 11 of each recess 5 has a circumferential undercut 12 and each liner 4a, 4b has a circumferential snap-fit lip 13 fitted to the undercut. This may of course also be embodied in the reverse. A slit 15 is arranged coaxially behind the snap-fit lips 13 so that the snap-fit lip 13 may snap into place.

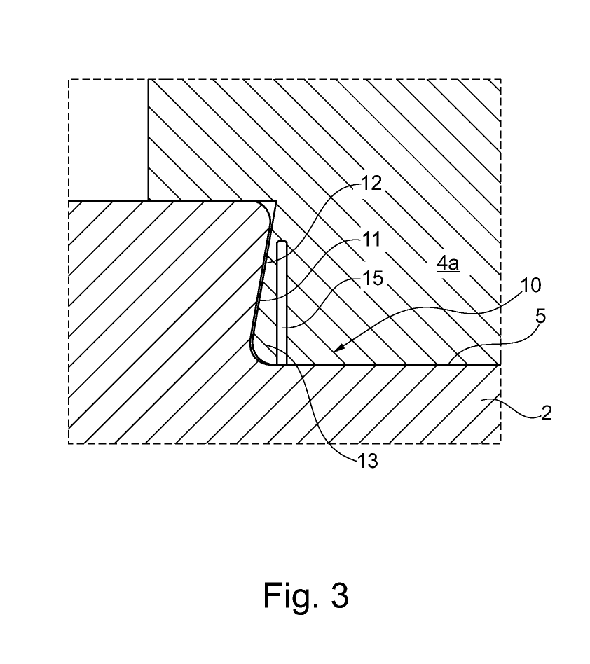

FIG. 3 depicts a detail from FIG. 2, specifically the snap-fit connection 10. The undercut 12 of each recess 5 facilitates the snap-fit lip 13 of the liner 4a snapping into place. Thus it is possible for a slit 15 to be arranged behind the snap-fit lip. Identical reference numbers always identify the same item.

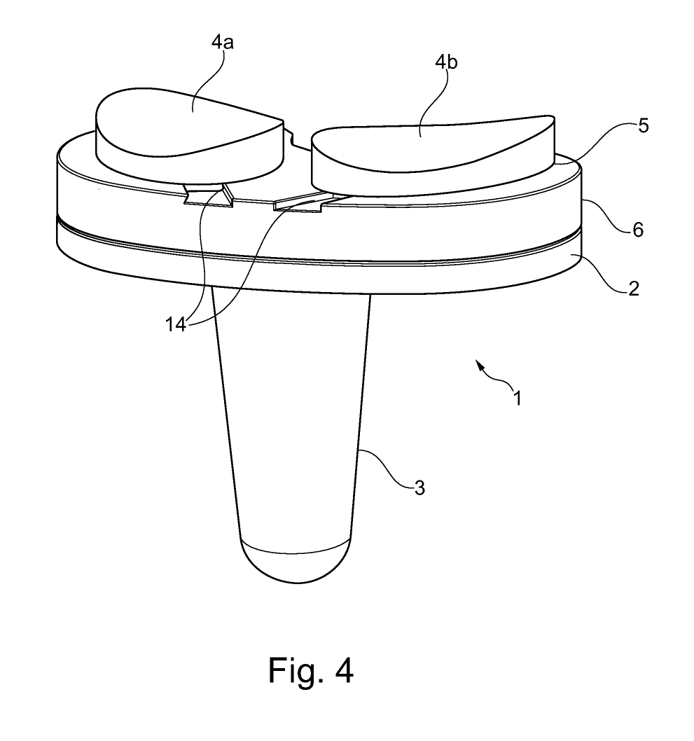

FIG. 4 depicts a second embodiment of the inventive tibial component 1 for anchoring to the proximal tibia.

A back-molded polymer 6 is arranged on the upper side of the tibial plateau 2 facing the femur part, on which back-molded polymer the liners 4a, 4b are each exchangeably anchored in a recess 5. In this embodiment, then, a back-molded polymer 6 is arranged between the tibial plateau 2 and the liners 4a, 4b. In this embodiment, as well, the liners 4a, 4b comprise UHMWPE, crosslinked PE, vitamin E PE, PEEK, or PAEK.

Arranged on the polymer 6 for each recess 5 is a ramp 14 like a slit, wherein the ramp 14 leads from the edge 11 of the polymer 6 to the floor of the recess 5 and because of this it is possible to guide the tip of a lever tool, known per se, under the liner 4a, 4b.

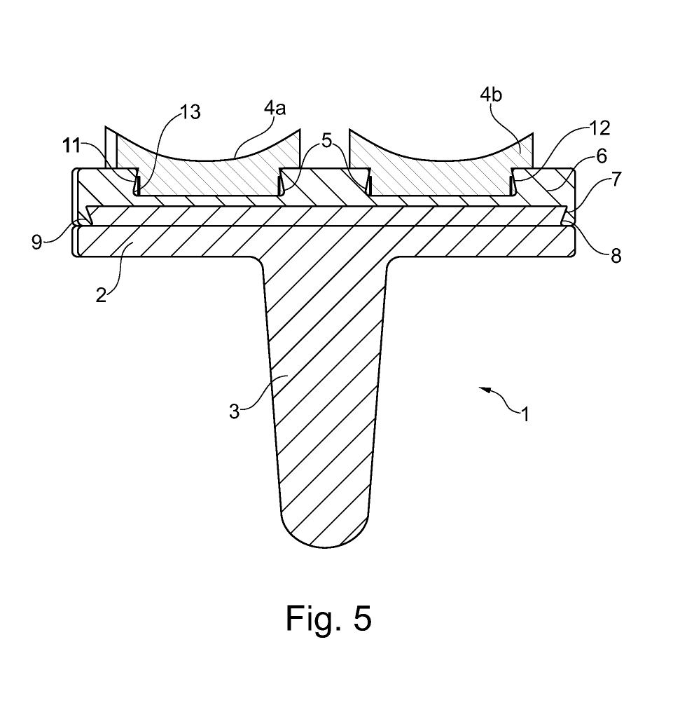

FIG. 5 depicts a section through the tibial component 1 according to FIG. 1. The circumferential edge 7 of the tibial plateau 2 has a notch 8 for anchoring the back-molded polymer 6 on the tibial plateau 2. A catch 9 of the back-molded polymer 6 is fitted to this notch 8 and engages in the notch 8. In this case, the liners 4a, 4b are anchored in the recesses 5 on the back-molded polymer 6 using a snap-fit connection 10.

To this end, the edge 11 of each recess 5 has a circumferential undercut 12 and each liner 4a, 4b has a snap-fit lip 13 fitted to the undercut 12. In this way this second embodiment is identical to the first embodiment, with the sole difference that the recess is arranged in the polymer 6 and not in the tibial plateau 2.

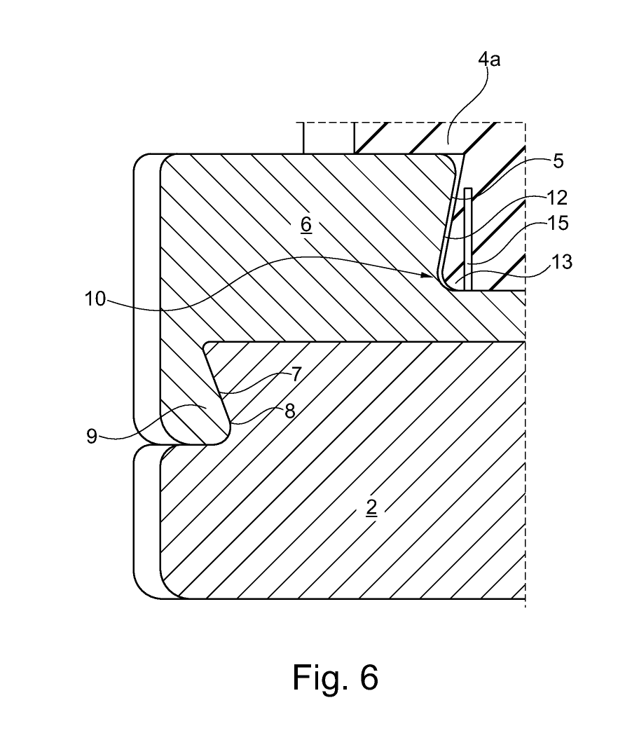

FIG. 6 depicts a detail of the tibial component according to FIGS. 4 and 5. It is evident that the circumferential edge 7 of the tibial plateau 2 is added to the notch 8. A catch 9 of the back-molded polymer 6 engages in this notch 8. It is also quite evident that arranged in the back-molded polymer 6 is a recess 5 having an undercut 12. The latter, together with the catch 13 of the liner 4a, forms a snap-fit connection 10. A slit 15 is arranged behind the catch 13 in this case, as well.

1. A tibial component of a knee endoprosthesis for anchoring on the proximal tibia, comprising a tibial plateau, a tibial shaft on the underside of the tibial plateau facing the tibia, and having a liner for receiving and supporting the lateral condyle surface of a femoral part, wherein the tibial component has one or, preferably, two liners embodied as individual parts that is/are exchangeably anchored on the tibial component.

2. The tibial component according to claim 1, wherein the liners comprise UHMWPE, crosslinked PE, vitamin E PE, PEEK, or PAEK.

3. The tibial component according to claim 1, wherein the tibial plateau and the tibial shaft comprise a metal or a sintered ceramic or even a polymer.

4. The tibial component according to claim 1, wherein for each liner a recess is arranged on the upper side of the tibial plateau facing the femur part, in which recess the liner is exchangeably anchored.

5. The tibial component according to claim 1, wherein a back-molded polymer is arranged on the upper side of the tibial plateau facing the femur part, on which back-molded polymer the liners are each exchangeably anchored in a recess.

6. The tibial component according to claim 5, wherein the circumferential edge of the tibial plateau has a notch in which a fitted catch of the back-molded polymer engages.

7. The tibial component according to claim 1, wherein the liners are anchored in the recesses on the tibial plateau or on the polymer using a snap-fit connection or press-fit connection.

8. The tibial component according to claim 7, wherein the edge of each recess has a circumferential undercut and each liner has a snap-fit lip fitted to the undercut.

9. The tibial component according to claim 1, wherein a ramp like a slit is arranged on the tibial plateau or on the polymer for each recess, the ramp leading from the edge of the tibial plateau or of the polymer up to the floor of the recess, this making it possible to guide the tip of a lever tool, known per se, under the liner.

10. A liner of a tibial component according to claim 1, wherein two liners are used for a bicondylar tibial base plate (revision tibial base plate) or only a single liner is used for a unicondylar tibial base plate.
